(19) United States
(12) Patent Application Publication
(10) Pub. No.: US 2015/0202181 A1
(43) Pub. Date: Jul. 23, 2015

(54) Method of and compounds for the reduction of alcoholic hangover

(71) Applicant: Paolo B. DePetrillo, North Potomac, MD (US)

(72) Inventor: Paolo B. DePetrillo, North Potomac, MD (US)

(73) Assignee: Paolo B DePetrillo, North Potomac, MD (US)

(21) Appl. No.: 13/999,170

(22) Filed: Jan. 23, 2014

(51) Int. Cl. A61K 31/385; A61K 31/198

(52) U.S. Cl. CPC A61K 31/385; A61K 31/198

(57) ABSTRACT

One embodiment of a composition containing 1200 mg of N-acetyl-L-cysteine and 400 mg of alpha-lipoic acid. For the purpose of eliminating many or all the symptoms of alcoholic hangover and the alcohol flushing reaction in susceptible individuals, this composition is ingested within 30 minutes prior to the initiation of drinking one or more alcoholic beverages and the same composition is ingested within 30 minutes after cessation of drinking alcoholic beverages.

### CROSS-REFERENCE TO RELATED APPLICATIONS

This application claims the benefit of provisional patent application Ser. No. 61/755,545, filed Jan. 23, 2013 by the present inventor.

### BACKGROUND

1. Field

This invention, relates to organosulfur compounds and more particularly, reduction of alcoholic hangover by administration of a composition of two organosulfur compounds and a method of administration of the formulation containing these two compounds.

2. Prior Art

The present invention comprises a novel composition and method for the reduction symptoms commonly called “alcoholic hangover” or just “hangover” by the oral delivery of a composition containing N-acetyl-L-cysteine (NAC) and alpha-lipoic acid (ALA) before and after a period of ethyl alcohol intake.

The experience of drinking an alcoholic beverage to excess, followed several hours later by highly unpleasant symptoms such as headache, nausea, vomiting, fatigue, heightened sensitivity to light and noise, weakness and thirst, vertigo, decreased attention span, and decreased concentration are unfortunately familiar to many people after imbibing beverages containing ethyl alcohol. We will use the term ‘alcohol’ to refer to ethyl alcohol. Accumulating evidence suggests that many of these symptoms may be due to the generation of acetaldehyde, a potent cellular toxin, during the metabolism of alcohol to acetate.

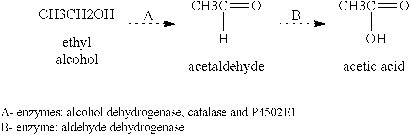

Ethyl alcohol is present in alcoholic beverages and is metabolized to acetaldehyde in the human body after oral ingestion. Ethyl alcohol is metabolized by several human enzymes generating acetaldehyde, a highly chemically reactive and toxic compound which is much more toxic than the parent compound ethyl alcohol. Acetaldehyde is chemically related to formaldehyde, which is the active agent in embalming fluid.

It is common knowledge in the fields of chemistry and biochemistry that acetaldehyde has an affinity for and can chemically combine with primary and secondary amines to form the corresponding imine or enamine respectively, negative biological function. Both proteins and DNA contain both primary and secondary amino groups. Both proteins and DNA are altered by acetaldehyde and may lose function. This chemical property is likely responsible for the preservative effect of acetaldehyde and formaldehyde on animal tissues.

The best-studied DNA adduct from acetaldehyde is N2-ethyl-2 2-deoxyguanosine, which is increased in liver DNA obtained from ethanol-treated rodents and in white blood cells obtained from human alcohol abusers. Studies of individuals who imbibe alcohol have clearly shown acetaldehyde-protein adducts as well. Acetaldehyde exposure after ethyl alcohol consumption may be a factor in the development of alcoholic liver disease, as well as high rates of digestive tract cancers occurring in Asian populations associated with alcohol consumption, because of the well-known prevalence of genetically determined slow acetaldehyde metabolism commonly present in individuals of Asian ancestry. This same genetic trait is also responsible for the well known flushing reaction which also occurs in these same individuals after ethyl alcohol consumption. A recent complete review of the role of acetaldehyde adducts in the genotoxicity of ethyl alcohol can be found in Brooks et al., Environ Mol Mutagen, doi: 10.1002/em.21824. A review of acetaldehyde-protein adducts and their contribution to liver disease can be found in Setshedi et al., Oxid Med Cell Longev, 3(3):178-185 (2010).

Acetaldehyde toxicity likely directly contributes to the subjective and objective experience of alcoholic hangover in humans. For example, a major risk factor for alcoholic hangover symptoms is a genetically determined decrease in aldehyde dehydrogenase activity as described by Yokoyama et al., Alcohol Clin Exp Res, 29(7):1165-1171 (2005).

A key premise of the present invention is that administration of compounds that act by decreasing the body burden of acetaldehyde resulting from alcohol metabolism after imbibing alcoholic beverages is a requirement for efficacy in the prevention of alcohol hangover symptoms. The mechanism-based search for and discovery of orally active non-toxic compounds that may be effective in decreasing physiological exposure to acetaldehyde after alcohol consumption distinguishes the present invention from much of the prior art.

The robust protective effects of several organosulfur compounds including L-cysteine and N-acetyl-L-cysteine against acetaldehyde toxicity in rodents was described by Sprince et al., Agents Actions, 5(2)164-175 (1975). Similar work by O'Neill et al., Res Commun Chem Pathol Pharmacol, 13(1):125-128 (1976) showed the same effect exerted by alpha-lipoic acid. A recent report by Li et al., Tohoku J Exp Med, 229(1):45-51 (2013)indicates that alpha-lipoic acid ameliorates oxidative stress by increasing aldehyde dehydrogenase activity in humans.

At least two compounds are naturally present in the human body which contain the correct chemical structure to easily react with acetaldehyde, and both contain thiol groups. The first is L-cysteine, an amino acid which is a component of body proteins, as well as glutathione. The reaction of acetaldehyde with L-cysteine occurs non-enzymatically and results in the synthesis of thiazolidinecarboxylic acid. N-acetyl-cysteine, (NAC) is an orally available precursor of the amino acid L-cysteine. L-cysteine itself is not well-absorbed after oral administration.

The second compound is called alpha-lipoic acid (ALA) shown below in its dehydrogenated form which is also a normal constituent of the human body. Lipoic acid exists in two enantiomeric forms, (R)-(+)-lipoic acid and (S)-(−)-lipoic acid. The term alpha-lipoic acid refers to the enantiomer found in nature, (R)-(+)-lipoic acid. Both of these organosulfur compounds act to decrease acetaldehyde levels by chemically combining with acetaldehyde in vivo, as shown below, before it can combine with cellular components such as proteins and DNA, thereby reducing or terminating their normal physiologic activities.

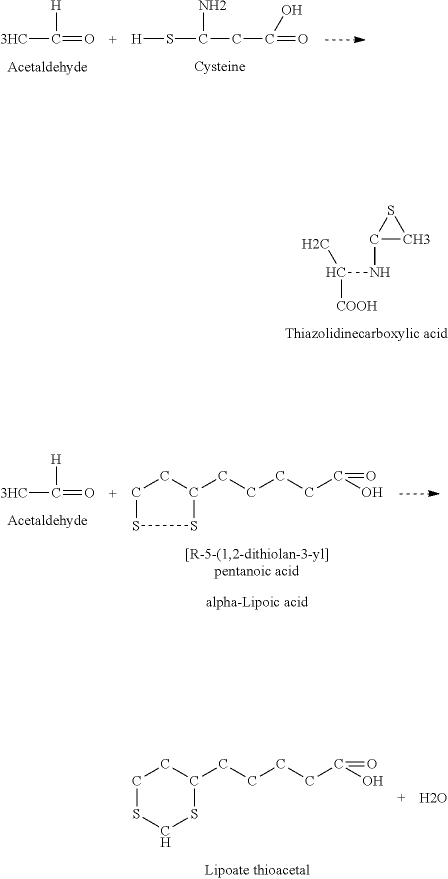

The prior art outlining treatments of alcohol hangover include ingestion of non-steroidal anti-inflammatory agents such as acetylsalicylic acid (Aspirin) and ibuprofen (Motrin, pain relievers such as acetaminophen, herbal-derived compounds with putative cytoprotective effects, various B-vitamins, and intravenous hydration with the addition of a potent steroid such as prednisone, as well as replacement of electrolytes such as sodium, potassium and magnesium.

The development of agents that can chemically sequester acetaldehyde was outlined in U.S. Pat. No. 6,686,336. The difference between the composition and method outlined heretofore is that the thiol-containing compounds described are xenobiotics, and hence may have additional side effects and or toxicities, as have been described for the parent compound, D-(−) penicillamine.

In the following continuing review of the prior art, the focus will be on those inventions which feature alpha-lipoic acid, N-acetyl-L-cysteine and L-cysteine, alone or in combination.

In US20070202215, the composition contains several vitamins, herbal extracts, and minerals, as well as L-cysteine at a dose of 12.5 to 37.5 mg. There is no mechanism proposed for inclusion of L-cysteine, but more importantly, it is well known that L-cysteine is poorly absorbed after oral ingestion, a factor which led to the development of N-acetyl-L-cysteine. L-cysteine is also proposed as a component in the compositions outlined in U.S. Pat. No. 4,496,548, in U.S. Pat. No. 5,202,354, in US20080057110 and in US20050271754.

In WO2012058589, the composition contains fructose, a variety of herbal extracts, 3-5 mg/ml N-acetyl-L-cysteine for a total of about 1000 mg in the proposed formulation, several B vitamins, lemon juice, acacia fiber and flavoring agents. The formulation proposes dilution of the composition in 240 ml liquid prior to oral intake. N-acetyl-L-cysteine is highly unpalatable in dissolved solutions.

In U.S. Pat. No. 8,377,907, the proposed composition contains 300 mg of N-acetyl-L-cysteine in addition to B-vitamins, Vitamin C, and glutathione as well as several other components. The proposed use of the composition is after, and not before, the beginning of ethyl alcohol consumption.

In US20080075710, one embodiment contains electrolytes, B-vitamins, silymarin, and N-acetyl-L-cysteine at a dose of 25 to 600 mg. The composition is ingested after, and not before, imbibing alcoholic drinks. In US20110171296, both L-cysteine and N-acetyl-L-cysteine are proposed as aldehyde binding agents in some of the embodiments, at a dose of up to 500 mg, taken after, and not before, the initiation of ethyl alcohol consumption. In U.S. Pat. No. 8,227,513, some of the embodiments contain N-acetyl-L-cysteine and/or L-cysteine. Several embodiments of the composition are added to water and foodstuff, but there are no directions relating to the timing of the use of the composition in relation to ethyl alcohol intake.

In WO2013009997, in addition to other agents, one of the embodiments includes N-acetyl-L-cysteine at a dose of up to 1000 mg. All embodiments are proposed to be consumed after, and not before, the consumption of beverages containing ethyl alcohol.

In US 20040248819, the composition might include fructose, a glucose-containing oligosaccharide, a branched chain amino acid, and alpha-lipoic acid, and a B-group vitamin. The rationale for including alpha-lipoic acid rests on the observation that this chemical is an anti-oxidant and may reduce reactive oxygen species generated by the metabolism of ethyl alcohol. However, it does not state the proposed effective dose. In EP1411879, the composition contains fructose, a fructose containing oligosaccharide, a branched-chain amino acid, and alpha-lipoic acid along with a B-vitamin. The amount of alpha-lipoic acid proposed for the composition is 10 mg. In our work with monotherapy of alcoholic hangover using alpha-lipoic acid, at least 200 mg must be ingested prior to imbibing alcoholic beverages for any noticeable reduction of alcohol hangover symptoms. In WO2012120036 describes and embodiment that containing both N-acetyl-L-cysteine at a dose of 700 mg and alpha-lipoic acid at a dose of 50 mg. The proportions of alpha-lipoic acid to N-acetyl-L-cysteine, 1:14 by weight is likely too small for prevention of alcoholic hangover.

### SUMMARY OF THE INVENTION

The present invention provides a composition and method of treatment of alcoholic hangover that is highly effective in reducing subjective symptoms of alcoholic hangover.

### DESCRIPTION OF PREFERRED EMBODIMENT

The formulation of the present composition contains both N-acetyl-L-cysteine (NAC) and alpha-lipoic acid (ALA).

A critical difference between NAC and ALA is that NAC, which is deacylated to L-cysteine, is more water soluble while ALA is more fat soluble. Since acetaldehyde is soluble in both lipid and water, it can partition into fatty tissue as well as body water. The combination of ALA and NAC results in better quenching of acetaldehyde than either compound alone because ALA will quench acetaldehyde in body spaces not fully accessible to NAC because of their high fat content, for example, the brain. Thus, the combination of NAC and ALA together achieve the objective of reducing hangover symptoms. When either compound, NAC or ALA, was tested as monotherapy, we found that much greater doses of either NAC or ALA were required to decrease alcohol hangover symptoms, making monotherapy with either NAC or ALA not feasible or practical. The dosing schedule and the frequency of dosing in timing relationship to alcohol consumption are also major issues in the use of these compounds for reducing hangover symptoms. For optimal reduction of alcoholic hangover symptoms, both compounds must be administered before and after the period of alcohol ingestion.

The molar weight of NAC is approximately 163, the molar weight of ALA is approximately 206, and the molar weight of alcohol is 46. In the United States, a standard drink of an alcoholic beverage contains 0.6 fluid ounces (18 milliliters) of ethyl alcohol. Thus, a standard drink contains approximately 0.3 moles of ethyl alcohol. The current formulation is a capsule containing 600 mg NAC and 200 mg ALA and a unit dose consists of two capsules of this composition. This represents on a molar basis approximately 0.02 moles capable of reacting with acetaldehyde. Both ethyl alcohol and the NAC/ALA are ingested concurrently, presenting much higher local concentrations of the protective composition in the stomach and liver. Ethyl alcohol metabolism in humans is a slow process and therefore it is important to dose with NAC/ALA both before and after a drinking session.

Based on preliminary open-label studies in humans drinking alcoholic beverages with NAC/ALA, we found a very large effect size; it is very unlikely to be to be due to a placebo effect. The relief of hangover symptoms is clear no matter in what form the alcoholic beverage is ingested, for example, beer or hard liquor, or if the alcoholic beverage or beverages are ingested with or without food.

We observed subjects of Asian ethnicity who reported that the composition of NAC/ALA robustly reduced or eliminated the alcohol-flushing reaction. This flushing reaction which is due to a lower metabolic rate for acetaldehyde in certain individuals of Asian descent. This finding supports the hypothesis that the novel composition of NAC/ALA was effective in reducing the body burden of circulating acetaldehyde and therefore the flushing reaction after ethyl alcohol ingestion.

In dose-finding studies, we determined that a combination of NAC and ALA, in a weight ratio of 3:1 NAC and ALA seems optimal for prevention of alcoholic hangover. Each capsule or tablet may be formulated to contain from 300 mg to 1200 mg of NAC and 100 mg to 400 mg ALA. The current formulation is a capsule containing 600 mg NAC and 200 mg ALA and the unit dose is two capsules. One unit dose is taken at the start of an alcohol drinking session, and one unit dose is taken at the end of the alcohol drinking session.

NAC is currently sold as a health supplement. Its major current medical use is to counteract acetaminophen (Tylenol) toxicity, for which its efficacy is widely recognized. It has a very high LD50 in rodents, and a human child survived with minor liver dysfunction seen after 8480 mg/kg/over 72 hours [http://chem.sis.nlm.nih.gov/chemidplus/rn/616-91-1]. ALA is also sold as a health supplement. The LD50 in rodents was >2000 mg/kg. Long-term safety is good, with the no-observed-effect level being 60 mg/kg/day. during chronic dosing for two years. It was found to be well-tolerated in diabetics given 600 mg/day for four years. Ziegler et al., Diabetes Care 34(9):2054-2060 (2011).

I claim:

1. A composition for treatment of alcoholic hangover comprising:
at minimum 1200 mg.of N-acetyl-L-cysteine per serving;
at minimum 400 mg of alpha-lipoic acid per serving.

2. A method of reduction of alcoholic hangover symptoms comprising the ingestion of one serving of the composition in accordance with claim 1 within 30 minutes of the beginning of a period of imbibing an alcoholic beverage and one serving within 30 minutes of the end of a period of imbibing an alcoholic beverage.

3. A method of reduction of the alcohol flushing reaction comprising the ingestion of one serving of the composition in accordance with claim 1 within 30 minutes of the beginning of a period of imbibing one or more alcoholic beverages and one serving within 30 minutes of the end of a period of imbibing one or more alcoholic beverages.
